(19) United States
(12) Patent Application Publication
(10) Pub. No.: US 2021/0087183 A1
(43) Pub. Date: Mar. 25, 2021

(54) PROCESS FOR THE PREPARATION OF OPICAPONE AND INTERMEDIATES THEREOF

(71) Applicant: UNICHEM LABORATORIES LTD, Maharashtra (IN)

(72) Inventors: Dhananjay G. SATHE, Maharashtra (IN); Arijit DAS, Goa (IN); Dnyaneshwar V. GAWAS, Goa (IN); Sanjay Bhaskar CHOWKEKAR, Mumbai (IN); Ravindra Subhash JAGTAP, Maharashtra (IN)

(21) Appl. No.: 16/954,501

(22) PCT Filed: Dec. 4, 2018
(86) PCT No.: PCT/IB2018/059598

§ 371 Date: Jun. 16, 2020

(30) Foreign Application Priority Data
Dec. 18, 2017 (IN) 201721045330

(51) Int. Cl. C07D 413/04

(52) U.S. Cl. CPC C07D 413/04

(57) ABSTRACT

The present invention is relates to a process for the preparation of opicapone and a process to prepare intermediates to be used therein.

### PRIORITY

This application claims the benefit of IN201721045330, file on Dec. 18, 2017, the content of which is incorporated herein by reference.

### FIELD OF INVENTION

The present invention is relates to a process for the preparation of opicapone and a process to prepare intermediates to be used therein.

### BACKGROUND OF THE INVENTION

Opicapone is a selective and reversible catechol-O-methyltransferase (COMT) inhibitor, use as adjunctive therapy for parkinson's disease. Opicapone was approved by European Medicine Agency (EMA) on Jun. 24, 2016 and it is developed and marketed as ONGENTYS® by Bial-Portela in Europe. Opicapone is chemically described as 2,5-dichloro-3-(5-(3,4-dihydroxy-5-nitrophenyl)-1,2,4-oxadiazol-3-yl)-4,6-dimethylpyridine-1-oxide and depicted below as compound of formula (I).

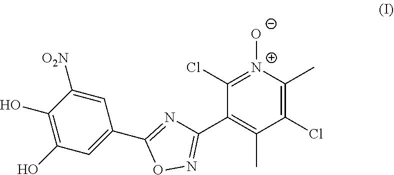 [SMILES: Cc1c(Cl)c(C)[n+]([O-])c(Cl)c1-c1noc(-c2cc(O)c(O)c([N+](=O)[O-])c2)n1]

Opicapone and a process for preparation of it is disclosed in U.S. Pat. No. 8,168,793. The process discloses condensation of 3,4-dibenzyloxy-5-nitrobenzoic acid with (Z)-2,5-dichloro-N′-hydroxy-4,6-dimethylnicotinimidamide in presence of N, N′-Carbonyldiimidazole in N, N′-dimethylformamide. The crude condensation intermediate was subjected to tetrabutylammonium fluoride (TBAF) mediated cyclization in tetrahydrofuran to give 1,2,4-oxadiazole derivative, purifying it by precipitating in 1:1 mixture of dichloromethane:diethyl ether and recrystallized it in isopropyl alcohol. Oxidation of 1,2,4-oxadiazole compound is carried out using 10 fold excess of urea hydrogen peroxide complex and trifluoroacetic anhydride in dichloromethane and was purified by column chromatography. Obtained N-oxide compound was converted into opicapone compound of formula (I) by deprotection O-benzoyl groups by exposure it to boron tribromide (BBr₃) in dichloromethane at −78° C. to room temperature. Final product was purified in mixture of toluene and ethanol. Above synthetic steps are outline in scheme 1.

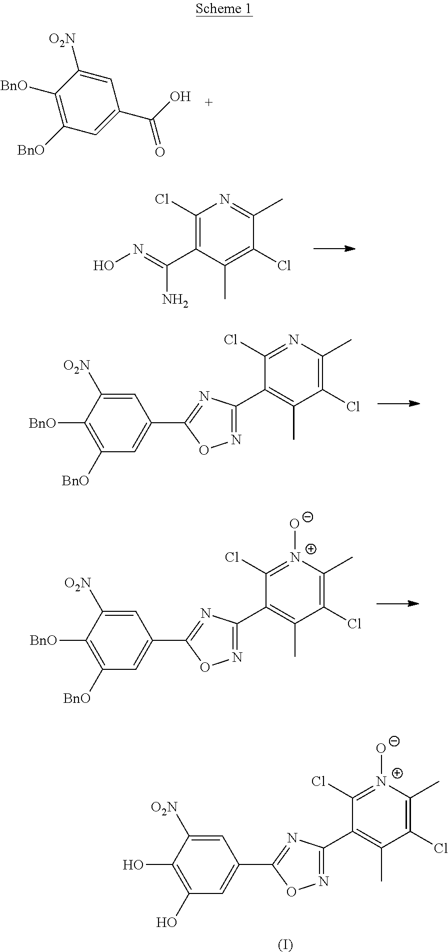

This process has several drawbacks like cyclization reaction involve use of TBAF and THF. Use of expensive TBAF, leads to high cost in the production and therefore uneconomical for industrial production, whereas use of THF during this reaction has limitation due to peroxide contents. Similarly diethyl ether is a potential fire hazard and can form peroxides rapidly and thus should be avoided in commercial scale production. Above cyclization is also carried out in presence of DMF and CDI at 120° C.

Similar approach was reported in WO2008094053 which describes preparation of opicapone by one pot cyclization of 3,4-dibenzyloxy-5-nitrobenzoic acid with (Z)-2,5-dichloro-N′-hydroxy-4,6-dimethylnicotinimidamide using N,N′-Carbonyl diimidazole in N,N′-dimethylformamide followed by heating the reaction mixture at 135° C. for 5 hours to obtain 1,2,4-oxadiazole derivative. This oxadiazole derivative was purified by recrystallization from isopropyl alcohol. Further oxidation using urea hydrogen peroxide complex followed by o-debenzylation using boron tribromide (BBr₃) was achieved to obtain Opicapone. This process also suffers from drawback like use of elevated temperature (135° C.) and use of expensive BBr₃.

U.S. Pat. No. 9,126,988 also disclose process for the preparation of opicapone, which involves several chemical steps: 1) nitrating vanillic acid in presence of nitric acid in acetic acid followed by recrystallization with acetic acid to get nitro compound with yield 40-46%; 2) which converted into acid chloride compound by treating it with thionyl chloride in presence of catalytic amount of N, N-dimethylformamide in dichloromethane or 1,4-dioxane; 3) condensing acid chloride compound with (Z)-2,5-dichloro-N′-hydroxy-4,6-dimethylnicotinimidamide in presence of excess amount of pyridine in N,N-dimethyl acetamide/tetrahydrofuran/dichloromethane or 1,4-dioxane at 5-10° C. and then heating the reaction mixture at 110-115° C. for 5-6 hours to get 1,2,4-oxadiazole compound; 4) which was oxidized using urea hydrogen peroxide complex and trifluoroacetic anhydride in dichloromethane to get N-oxide product which was purified by repeated recrystallization (2 or more times) using mixture of formic acid and toluene to get pure product with 59% yield; 5) O-methyl group was deprotected using aluminium chloride and pyridine in N-Methyl pyrrolidone at 60° C. to obtain opicapone. After completion of reaction, the crude product was isolated by quenching the reaction mixture in mixture conc. HCl:water followed by filtration, washing with water:isopropyl alcohol and recrystallization from ethanol. Final purification was done in mixture of formic acid and isopropyl alcohol. Above synthetic steps are outline in scheme 2.

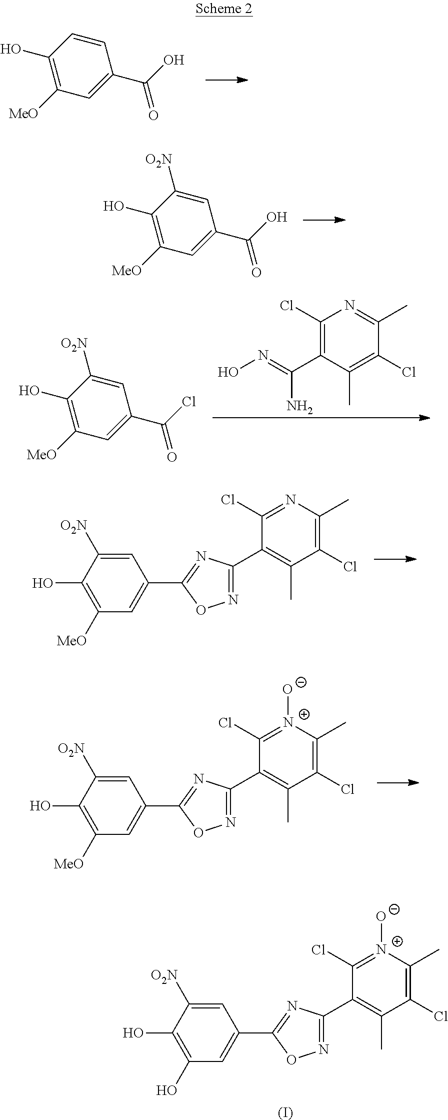

As described above, cited literature processes suffers from some drawbacks like elevated reaction temperature and longer duration, use of excess amount of pyridine for cyclization reaction which is difficult to handle on large scale preparation. Another drawback of reported procedure is unsafe workup procedures for isolation of N-oxide as residual peroxides were not quenched by any peroxide quenching reagent. Also repeated crystallizations (more than two) are required for purification of N-oxide derivative to remove unreacted starting material which is tedious and time consuming process. Also for its purification mixture of solvent i.e. formic acid and toluene are used which hamper its recovery and is not cost effective process.

U.S. Pat. No. 9,126,988 also disclosed process for the preparation of 2,5-dichloro-N′-hydroxy-4,6-dimethylnicotinimidamide compound of formula (IV), in which 2,5-dichloro-4,6-dimethylnicotinonitrile compound of formula (VIII) was reacted with hydroxyl amine solution in the presence of catalytic amount of 1,10-phenanthroline in methanol:water at 70-80° C. for 6 hrs. After completion reaction mixture was cooled, filtered and dried to get 2,5-dichloro-N′-hydroxy-4,6-dimethylnicotinimidamide of formula (IV) (88%).

Bioorganic & Medicinal Chemistry 13 (2005) 5740-5749, Karl Bailey et. al. disclosed process for preparation of 3,4-dimethoxy-5-nitro benzoic acid compound of formula (IIIa). In which a solution of CrO₃, concentrated H₂SO₄ and water was added to solution of 3,4-dimethoxy-5-nitro benzaldehyde in acetone and water. The obtained solution was stirred for 24 hrs and then isopropanol was added to eliminate any unreacted Cr(VI) species to obtained crude green sludge, which was extracted into ethyl acetate and washed with 1M HCl to remove remaining Cr(III) species. Obtained product is then recrystallized from water and ethanol to yield 69% of 3,4-dimethoxy-5-nitro benzoic acid compound of formula (IIIa).

U.S. Pat. No. 5,358,948 also disclosed process for preparation of 3,4-dimethoxy-5-nitro benzoic acid compound of formula (IIIa). In which a solution of potassium permanganate was added to a solution of 3,4-dimethoxy-5-nitro benzaldehyde in acetone. The mixture was then stirred at 20° C. for 18 hrs to gives 3,4-dimethoxy-5-nitro benzoic acid compound of formula (IIIa) with 72% yield.

Disadvantage of the above cited literature (Karl Bailey et. al and U.S. '948) processes are harsh, acidic condition and involve expensive reagents. The process is both uneconomical and time consuming, (18-24 hrs) hence not suitable for commercial production.

Oxidation of aldehydes to the corresponding carboxylic acids, on the other hand, are commonly carried out using KMnO4 in acidic or basic media, or K2Cr2O7 in acidic medium or chromic acid. These heavy metal-based reagents are hazardous and the protocols produce metal wastes that require special handling owing to their toxicities.

It is therefore, desirable to provide efficient, robust, alternative simple process, cost effective process which is used on a large scale and allows product to be easily workup, purified and isolate without the disadvantages mentioned above.

### OBJECT OF THE INVENTION

An object of the invention is to provide process for the preparation of opicapone, overcoming the defects and deficiencies in the prior literatures.

Yet another object of the invention is to provide intermediates and process thereof for the preparation of opicapone.

Yet another object of the invention is to provide process for the preparation of compound of formula (IV).

Yet another object of the invention is to provide process for the preparation of compound of formula (III).

Yet another object of the invention is to provide process for the purification of compound of formula (VII) or (VIa) using Bronsted acid.

Yet another object of the invention is to provide method for purification of opicapone in presence of organic solvent

### SUMMARY OF THE INVENTION

An object of the present invention relates to process for the preparation of opicapone compound of formula (I) comprising;
a) reacting compound of formula (II) with oxone to obtain compound of formula (III);
b) reacting compound of formula (III) with compound of formula (IV) to obtain compound of formula (V);
c) cyclizing compound of formula (V) to obtain compound of formula (VI);
d) oxidizing compound of formula (VI) with oxidizing agent to produce compound of formula (VII); and
e) deprotecting hydroxyl protecting group of compound of formula (VII) to obtain opicapone compound of formula (I)
   wherein R₁ and R₂ independently from each other represent hydrogen or a suitable protecting groups for aromatic hydroxyl groups.

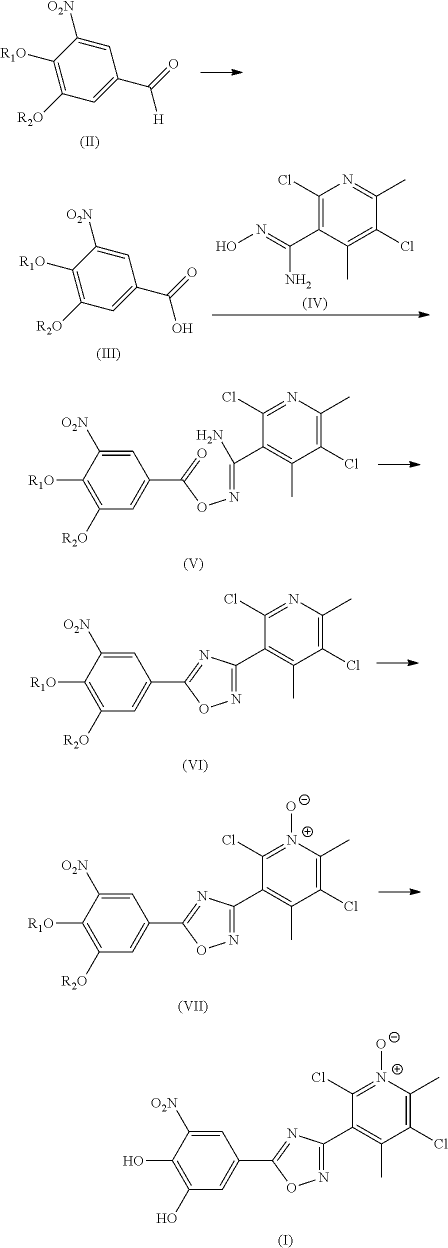

The present invention also relates to provide intermediates of compound of formula (Va), (VIa) and (VIIa) for the preparation of opicapone compound of formula (I).

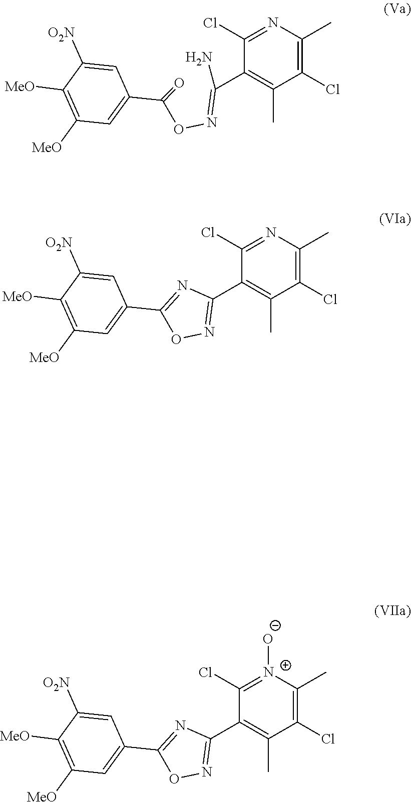 [SMILES: COc1cc(C(=O)O/N=C(\N)c2c(Cl)nc(C)c(Cl)c2C)cc([N+](=O)[O-])c1OC ; COc1cc(-c2nc(-c3c(Cl)nc(C)c(Cl)c3C)no2)cc([N+](=O)[O-])c1OC ; COc1cc(-c2nc(-c3c(C)c(Cl)c(C)[n+]([O-])c3Cl)no2)cc([N+](=O)[O-])c1OC]

Yet another object of the invention is to control formation of impurities like impurity A, impurity B and impurity C during preparation of opicapone.

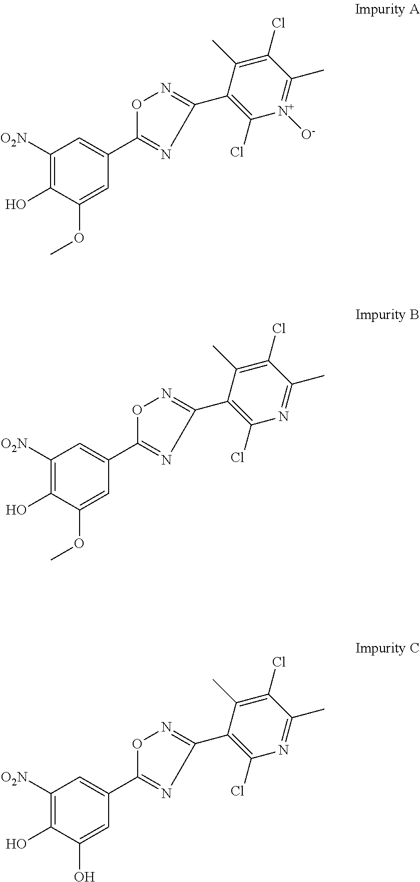 [SMILES: COc1cc(-c2nc(-c3c(C)c(Cl)c(C)[n+]([O-])c3Cl)no2)cc([N+](=O)[O-])c1O ; COc1cc(-c2nc(-c3c(Cl)nc(C)c(Cl)c3C)no2)cc([N+](=O)[O-])c1O ; Cc1nc(Cl)c(-c2noc(-c3cc(O)c(O)c([N+](=O)[O-])c3)n2)c(C)c1Cl]

Yet another object of the invention is to provide process for the preparation of compound of formula (IV) wherein process comprises reacting compound of formula (VIII) with hydroxyl amine or salt thereof in the presence of base.

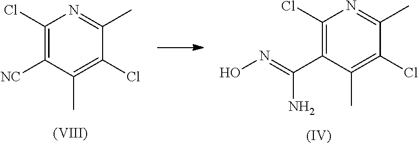

Yet another object of the invention is to provide process for the preparation of compound of formula (III) by oxidizing compound of formula (II) using oxone.

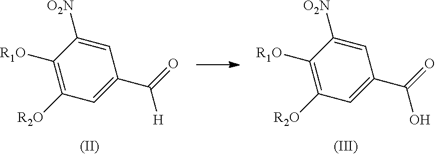

wherein R₁ and R₂ defined as above.

Yet another object of the invention is to provide process for the purification of compound of formula (VII) or (VIIa) using Bronsted acid in presence of organic solvent.

Yet another object of the invention is to provide method for purification of opicapone in presence of organic solvent.

### DETAILED DESCRIPTION OF THE INVENTION

The present invention provides a process for the preparation of opicapone compound of formula (I) comprising;
a) reacting compound of formula (II) with oxone to obtain compound of formula (III);

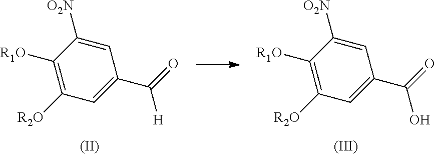

b) reacting compound of formula (III) with compound of formula (IV) to obtain compound of formula (V);

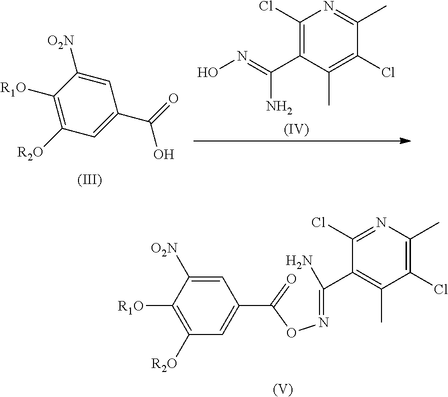

c) cyclizing compound of formula (V) in presence of base to obtain compound of formula (VI);

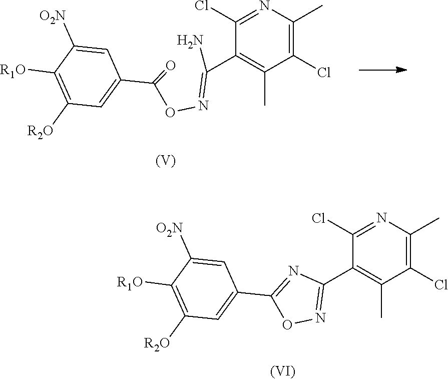

d) oxidizing compound of formula (VI) with oxidizing agent to produce compound of formula (VII); and

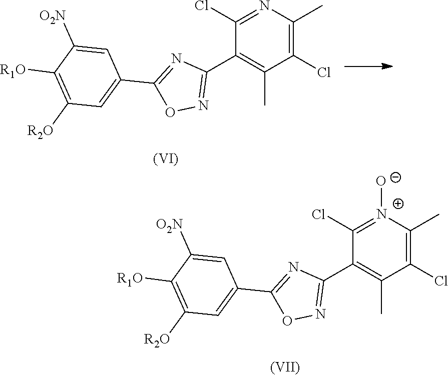

e) deprotecting hydroxyl protecting group of compound of formula (VII) to obtain opicapone compound of formula (I);

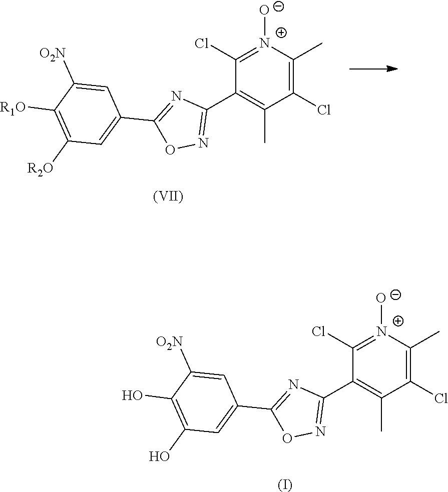

wherein R₁ and R₂ independently from each other represent hydrogen or a suitable protecting groups for aromatic hydroxyl groups.

The specific reaction schemes is as follows:

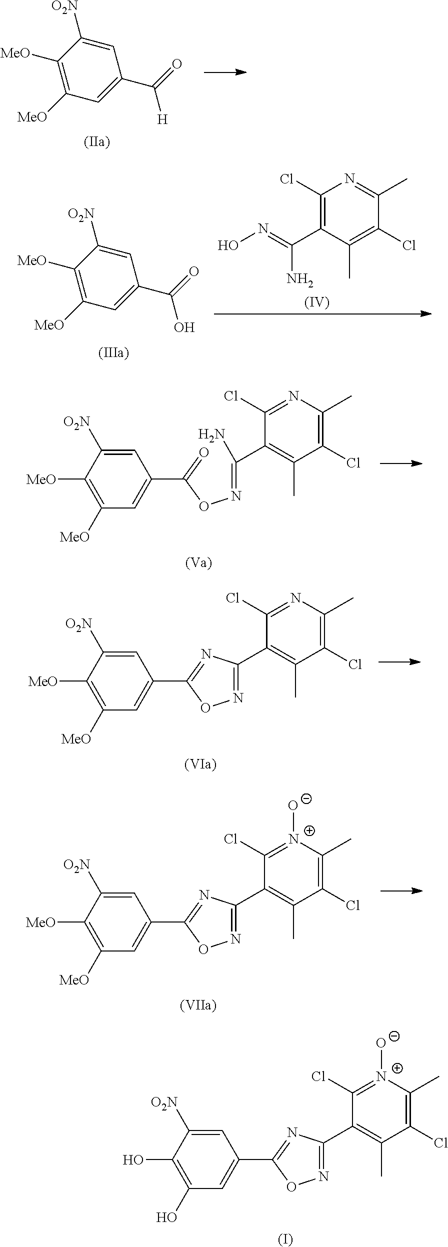

Suitable protective groups for aromatic hydroxyl groups are well known in the art. Examples of suitable protective groups for aromatic hydroxyl groups include but not limited to methyl, ethyl, isopropyl, butyl, benzyl, 4-methoxybenzyl, methoxymethyl, benzyloxymethyl, methoxyethoxymethyl, tetrahydropyranyl, phenacyl, allyl, trimethylsilyl, tert-butyldimethylsilyl, benzyloxycarbonyl, tert-butoxycarbonyl, ester, sulphonate, carbamate, phosphinate, acetal, ketal derivatives and the like.

### Step (a):

The oxidation of compound of formula (II) or (IIa) with oxone is carried out in presence of solvent selected from group consisting of N,N-Dimethylmethanamide (DMF), acetone, acetonitrile, N-methylpyrrolidone (NMP), Hexamethylphosphoramide (HMPA), pyrrolidinone, Tetrahydrofuran, water, ethyl acetate, 1,4-dioxane, acetonitrile, propionitrile, acetone, ethyl methyl ketone, formamide, chlorinated hydrocarbons such as dichloromethane, ethylene dichloride, chloroform, dimethyl acetamide, propionamide, nitromethane, 1,2-dimethoxyethane, 2-methoxyethanol, 2-ethoxy ethanol, aliphatic and alicyclic hydrocarbons such as hexane, heptane, pentane, cyclohexane, methyl cyclohexane, aliphatic esters and mixture(s) thereof. More preferably DMF.

The reaction is carried out for about 1 to 10 hrs at 0° to 50° C., Obtained product is isolated by addition of excess of water followed by filtration and used in the next step with or without purification.

Purification of crude acid derivative is done by giving water slurry to remove excess of peroxide content followed by filtration and drying.

Above oxidation reaction facile, high yielding, and easy to workup, less time required to compete the reaction and should provide a mild oxidation alternative for CrO₃, concentrated H₂SO₄, potassium permanganate.

Oxone, commercially available from Aldrich Chemical Company, is a 2:1:1 molar mixture of KHSO5, KHSO4, and K2SO4 and is readily soluble in water. Considering the water-solubility and the environmentally safe and benign nature of Oxone, we recently used this reagent in conjunction with another oxidant for the oxidation of alcohols to carboxylic acids.

### Step (b):

The condensation of compound of formula (III) or (IIIa) with compound of formula (IV) is carried out in presence of condensing agent and suitable solvent at temperature 0° C. to 30° C. depending on boiling point of the utilized solvent system. The reaction temperature preferably at room temperature. The reaction time is in the range of 30 minutes to 24 hours.

The condensing agents used for reaction are selected from group consisting of N, N′-Carbonyldiimidazole, thionyl chloride, sulfonylchloride, N,N′-dicyclohexylcarbodiimide, 1-hydroxybenzotriazole and N-(3-dimethylaminopropyl)-N′-ethylcarbodiimide, phosgene, PCl3, POCl3, PCl5, anhydrides, trichlorotriazine and chlorodimethoxytriazine and the like.

The condensation of step (b) is performed in an organic solvent selected from dimethylformamide, dimethylsulfoxide, dimethylacetamide and N-methylpyrrolidinone, acetonitrile, Tetrahydrofuran, ethyl acetate, 1,4-dioxane, acetonitrile, propionitrile, acetone, ethyl methyl ketone, formamide, chlorinated hydrocarbons such as dichloromethane, ethylene dichloride, chloroform, dimethyl sulphoxide, sulpholane, acetamide, propionamide, nitromethane, anisole, aliphatic and alicyclic hydrocarbons such as hexane, heptane, pentane, cyclohexane, methyl cyclohexane, aliphatic esters and aromatic hydrocarbons such as toluene, mixture of xylenes and mixture(s) thereof.

The compound of formula (V) or (Va) is isolated by addition of excess of water followed by filtration and drying.

### Step (c):

Cyclization of compound of formula (V) or (Va) is carried out at room temperature in presence of base and organic solvent to obtain compound of formula (VI) or (VIa). The reaction time is in the range of 30 minutes to 24 hours.

Cyclization is carried out in presence of organic solvent selected from methanol, ethanol, propanol, isopropanol, n-butanol, tert-butanol, tetrahydrofuran, water, 1,4-dioxane, acetonitrile, propionitrile, acetone, ethyl methyl ketone, formamide, N,N, dimethylformamide, chlorinated hydrocarbons such as dichloromethane (MDC), ethylene dichloride, chloroform, dimethyl sulphoxide, sulpholane, acetamide, propionamide, nitromethane, 1,2-dimethoxyethane, 2-methoxyethanol, 2-ethoxy ethanol, anisole, aliphatic and alicyclic hydrocarbons such as hexane, heptane, pentane, cyclohexane, methyl cyclohexane, aliphatic esters, aromatic hydrocarbons such as toluene, mixture of xylenes and/or mixture(s) thereof.

The base used in cyclization reaction are organic bases such as triethyl amine, diisopropylethylamine, DMAP, and/or aqueous solution(s) and mixture(s) thereof and inorganic bases such as sodium hydroxide (NaOH), potassium hydroxide (KOH), potassium carbonate, sodium carbonate, sodiumbicarbonate, potassium bicarbonate, sodium hydride and/or and aqueous(s) solution and mixture(s) thereof; other bases such as potassium tert-butoxide, sodium tert-butoxide and/or and aqueous(s) solution and mixture(s) thereof.

In an alternative object of the present invention is to provide one pot process to prepare compound of formula (VI) or (VIa) by condensation and cyclization reaction is carried out in the same reaction vessel. In which condensation of compound of formula (III) or (IIIa) with compound of formula (IV) is carried out in presence of condensing agent as described in above (step (b)), followed by cyclization of compound of formula (V) or (Va) in presence of base as described in above. In this one pot process, condensation and cyclization are conducted sequentially in the same reaction vessel without isolation of compound of formula (V) or (Va).

Yet another objective of the present invention is to use alkyl group protection for both phenolic hydroxy group in compound of formula (IIIa). The alkyl protection of both phenolic hydroxy group in compound of formula (Ia) becomes very advantageous because one can remove all acidic impurities present in the compound of formula of (VIa) or (VIIa) by simple washing with basic aqueous solution during work up procedure.

Cyclization step in present invention for preparation of 1,2,4-oxadiazole derivative involves simple reaction conditions such as ambient reaction temperature and use of cheaper inorganic base such as KOH or NaOH making process simple and cost effective.

### Step (d):

Oxidation reaction of compound of formula (VI) or (VIa) is performed with oxidising agent. N-oxide group can be introduced to compound of formula (VI) or (VIa) by using oxidizing agent such as hydrogen peroxide, MnO₂, peracetic acid, trifluoroperacetic acid, t-butylhydroperoxide, m-chloroperoxybenzoic acid, persulfuric acids, Oxone®, urea hydrogen peroxide complex and trifluoroacetic anhydride, pyridinium chlorochromate and permanganate ions. Preferred oxidation is performed with urea hydrogen peroxide complex in presence of organic acid anhydride such as trifluoroacetic anhydride.

The solvent used in oxidation step (d) is selected from halogenated solvents, such as dichloromethane, chloroform, chlorobenzene and carbon tetrachloride, aromatic solvents such as benzene and toluene, alkanes such as cyclohexane and hexane, and ethers such as THF, 1,4-dioxane diisopropyl ethyl ether, cyclopentyl methyl ether and tert-butylmethylether and mixture(s) thereof and other solvents are formic acid, acetic acid, trifluoroacetic acid, DMF, N,N-dimethylacetamide (DMA), and mixture(s) thereof.

The oxidation reaction of step (d) is performed at temperature is in range from 5° C.-100° C., during reaction and workup process for about 1 to 24 hours.

Obtained N-oxide derivative compound of formula (VII) or (VIIa) is purified by using organic solvent selected from acetone, toluene, ethyl acetate, dichloromethane, chloroform, carbontetrachloride, methanol, ethanol, isopropyl alcohol, n-propanol, n-butanol, DMF, dimethyl sulfoxide (DMSO), DMA, NMP, Bronsted acids such as HCl, H₂SO₄, HBr, acetic acid, formic acid, trifluoroacetic acid and/or mixture(s) thereof. More preferably N-oxide derivative was purified by using conc. hydrochloric acid and ethyl acetate.

N-oxide derivative is treated with conc. hydrochloric acid in ethyl acetate at 25-80° C. followed by cooling to room temperature and filtration to obtain compound of formula (VII) or (VIIa)

The reported procedure in U.S. Pat. No. 9,126,988 does not mention any quenching protocol for residual peroxides at N-oxide preparation stage which is serious limitation as peroxide may explodes when dry. Yet another objective of the present invention is to use alkyl group protection for both phenolic hydroxy group in compound of formula (VIIa) which is advantageous to use peroxide quenching reagents which are basic in nature.

### Step (e):

The deprotection of both phenolic hydroxy protecting groups of compound of formula (VII) or (VIIa) is performed in a suitable physiological condition in organic solvent.

The deprotection is carried out in the presence of Lewis acid, Bronsted acids such as HCl, H₂SO₄, HBr, acetic acid, formic acid, trifluoroacetic acid, Pd/c, AlCl₃.

In the preferred embodiment of the present invention; deprotection of hydroxy protecting group of compound of formula (VII) or (VIIa) is carried out in presence of aluminium chloride (AlCl3) in organic solvent such as N, N-dimethyl formamide at temperature is in range temperature range from 5-120° C. The present invention developed cost effective process by avoiding use of pyridine which creates complications during work up procedure.

The organic solvent selected from toluene, ethyl acetate, xylenes, DMF, DMSO, MDC, NMP and/or mixture(s) thereof.

Opicapone compound of formula (I) is further purified by using organic solvent selected from methanol, ethanol, isopropyl alcohol, DMF, DMSO, DMA, NMP, acetic acid, and/or mixture(s) thereof.

HPLC analysis of opicapone showed absences of impurity A, impurity B & impurity C.

Yet another object of the invention is to provide the intermediate compound of the formula (VIa), (VIIa) and (VIIIa) and the process for the preparation of same as described above.

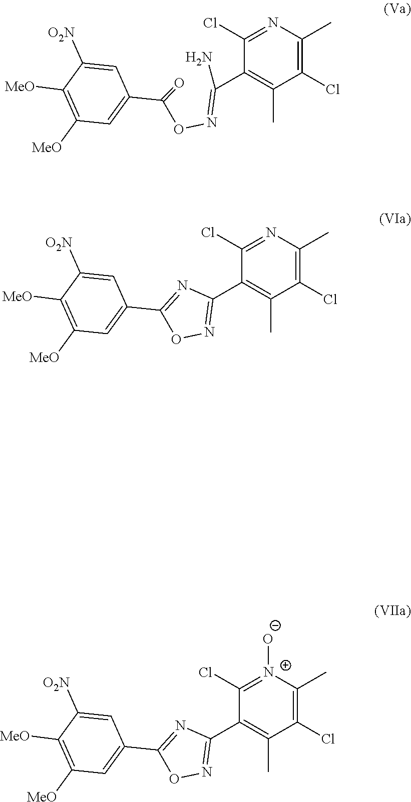 [SMILES: COc1cc(C(=O)O/N=C(\N)c2c(Cl)nc(C)c(Cl)c2C)cc([N+](=O)[O-])c1OC ; COc1cc(-c2nc(-c3c(Cl)nc(C)c(Cl)c3C)no2)cc([N+](=O)[O-])c1OC ; COc1cc(-c2nc(-c3c(C)c(Cl)c(C)[n+]([O-])c3Cl)no2)cc([N+](=O)[O-])c1OC]

Yet another object of the invention is to provide process for the preparation of compound of formula (IV) comprising reacting compound of formula (VIII) with hydroxyl amine or salt thereof in the presence of base.

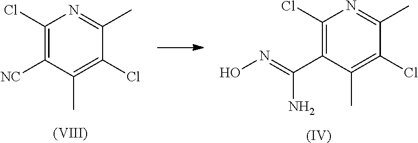

The reaction of compound of formula (VIII) was carried out using hydroxylamine in the presence of catalytic or stoichiometric amount of organic base such as pyridine, triethyl amine, N,N,N′,N′-tetramethylethylenediamine, diisopropylethyl amine, 4-dimethylaminopyridine, N-methyl morpholine, pyrazine or its derivatives (2-methyl pyrazine, 2,5-dimethyl pyrazine) and/or aqueous thereof.

The reaction of compound of formula (VIII) was carried out using hydroxylamine salts in the presence of inorganic base such as LiOH, KOH, NaOH, K₂CO₃, Na₂CO₃, Li₂CO₃, NaHCO₃, KHCO₃ and catalytic amount of organic base such as pyridine, triethyl amine, N,N,N′,N′-tetramethylethylenediamine, diisopropylethyl amine, 4-dimethylaminopyridine, N-methyl morpholine, pyrazine or its derivatives and/or aqueous thereof.

The solvent used in the above reaction is selected from methanol, ethanol, isopropanol, n-propanol, n-butanol, tert-butanol, DMF, DMSO, NMP, acetonitrile, tetrahydrofuran, 1,4-dioxane, water and/or mixture(s) thereof.

The reaction is carried out at temperature range from 25°-90° C. The reaction time is in the range of 5-10 hours.

The example of hydroxylamine salt is selected from hydrochloride, hydrobromide, sulfate salts.

The present invention use of commercially cheap hydroxyl amine salts for preparation of compound of formula (IV) to make cost effective process.

Yet another object of the invention is to provide process for the preparation of compound of formula (III) as described in step (a) as above.

Purification of Opicapone is carried out in presence of organic solvent selected from methanol, ethanol, isopropyl alcohol, dichloromethane, Tetrahydrofuran, toluene, N,N-dimethylformamide, Dimethylsulfoxide, N,N-dimethylacetamide, N-methyl-2-pyrrolidone, acetic acid, ethylacetate, acetone, and mixture(s) thereof. More preferably Opicapone of formula (I) was purified using mixture of N,N-dimethylformamide and methanol to obtain compound of formula (I).

### EXAMPLES

The following examples are presented for illustration only, and are not intended to limit the scope of the invention or appended claims.

### Example 1: Preparation of 3,4-dimethoxy-5-nitro Benzoic Acid (IIIa)

To a cooled solution of 3,4-dimethoxy-5-nitro benzaldehyde (100 g, 0.474 mole) in DMF (500 ml) was added Oxone (294.1 g, 0.478 mole) lot wise at 5-10° C. Reaction mixture was stirred for 30 minutes at same temperature, allowed to warm to room temperature and stirred for 2-3 hours. After completion, the reaction mixture was diluted with 1500 ml of water and filtered. The solid was washed with water until all peroxides removed and drying at 50° C. under vacuum afforded 3,4-dimethoxy-5-nitro benzoic acid of formula (IIIa) (102 g, 95%).

### Example 2: Preparation of 2,5-dichloro-N′ {[(3,4-dimethoxy-5-nitrophenyl) carbonyl]oxy}-4,6-dimethylpyridine-3-carboximidamide (Va)

To a solution of 3,4-dimethoxy-5-nitro benzoic acid of formula (IIIa) (5 g, 0.022 mole) in 60 ml of acetonitrile was added N,N′-Carbonyldiimidazole (4.28 g, 0.026 mole) in portions and the reaction mixture was stirred at room temperature for 1.5 hours. Then was added 2,5-dichloro-N′-hydroxy-4,6-dimethylnicotinimidamide of formula (IV) (5.4 g, 0.023 mole) and stirring was continued for 3 hours. After completion, the reaction mixture was diluted with 240 ml of water and 300 ml of dichloromethane. Organic layer was separated and washed with water (200 ml×3), concentrated under reduced pressure to obtain 2,5-dichloro-N′{[(3,4-dimethoxy-5-nitrophenyl)carbonyl]oxy}-4,6-dimethylpyridine-3-carboximidamide of formula (Va) (8.67 g, 88.9%).

### Example 3: Preparation of 2,5-dichloro-3-[5-(3,4-dimethoxy-5-nitrophenyl)-1,2,4-oxadiazol-3-yl]-4,6-dimethylpyridine (VIa)

To a solution of 2,5-dichloro-N′{[(3,4-dimethoxy-5-nitrophenyl)carbonyl]oxy}-4,6-dimethylpyridine-3-carboximidamide of formula (Va) (0.5 g, 0.0011 mole) in 10 ml of dichloromethane was added isopropyl alcohol (1 ml) followed by KOH (0.075 g, 0.0011 mole) dissolved in 0.1 ml of water. After stirring for 1 hour at room temperature the reaction mixture was diluted with 30 ml of dichloromethane and washed with water (10 ml×2). The reaction mixture was concentrated under reduced pressure to obtain 2,5-dichloro-3-[5-(3,4-dimethoxy-5-nitrophenyl)-1,2,4-oxadiazol-3-yl]-4,6-dimethylpyridine of formula (VIa) (0.4 g, 83%).

### Example 4: Preparation of 2,5-dichloro-3-[5-(3,4-dimethoxy-5-nitrophenyl)-1,2,4-oxadiazol-3-yl]-4,6-dimethylpyridine (VIa) (One Pot Cyclization Procedure)

To a stirred solution of 3,4-dimethoxy-5-nitro benzoic acid formula (IIIa) (100 g, 0.44 mol) in 1000 ml of dichloromethane was added N,N′-Carbonyldiimidazole (86 g, 0.53 mole) in portions and the reaction mixture was stirred at room temperature for 1.5 hours. Then was added 2,5-dichloro-N′-hydroxy-4,6-dimethylnicotinimidamide of formula (IV) (108 g, 0.46 mole) and stirring was continued for 3 hours. Isopropyl alcohol (200 ml) and KOH (30 g, 0.53 mole) dissolved in 30 ml of water was then added to the reaction mixture. After stirring for 1 hour at room temperature the organic layer was washed with water (1000 ml×2). Solvent was distilled out at atmospheric pressure, added 1000 ml of isopropyl alcohol and suspension was stirred at 55-60° C. for 2 hours. The reaction mixture was allowed to cool to room temperature, stirred for 2 hours and filtered. The solid was washed with isopropyl alcohol (100 ml×2) and dried at 50-60° C. under vacuum to obtain 2,5-dichloro-3-[5-(3,4-dimethoxy-5-nitrophenyl)-1,2,4-oxadiazol-3-yl]-4,6-dimethylpyridine of formula (VIa) (160 g, 85%).

### Example 5: Preparation of 2,5-dichloro-3-[5-(3,4-dimethoxy-5-nitrophenyl)-1,2,4-oxadiazol-3-yl]-4,6-dimethylpyridine (VIa) (Cyclization Procedure Using Thionyl Chloride)

To a stirred solution of 3,4-dimethoxy-5-nitro benzoic acid of formula (IIIa) (100 g, 0.44 mol) in 500 ml of dichloromethane was added 0.4 ml of N,N-dimethyl formamide followed bythionyl chloride (82 g, 0.69 mole) drop wise at room temperature and the reaction mixture was heated at 40° C. for 4 hours. After completion, dichloromethane and excess of thionyl chloride was distilled out under reduced pressure at 40° C. The obtained residue was dissolved in 500 ml of dichloromethane and was added to pre-cooled mixture of 2,5-dichloro-N′-hydroxy-4,6-dimethylnicotinimidamide of formula (IV) (103 g, 0.44 mole) and triethyl amine (73 ml, 0.53 mole) in 500 ml of dichloromethane at 5° C. After addition, the reaction mixture was allowed to warm to 25-30° C. and stirred for 2 hours. Then was added isopropyl alcohol (200 ml) followed by KOH (62 g, 1.1 mole) dissolved in 62 ml of water and stirring was continued for 2 hours at room temperature. The reaction mixture was washed with 1000 ml of water, 1N aqueous HCl solution (500 ml×2) followed by 500 ml of 5% aqueous sodium bicarbonate solution. Solvent was distilled out at atmospheric pressure at 40° C. To the residue was added 1200 ml of methanol and the suspension was stirred at 55-60° C. for 2 hours. The reaction mixture was allowed to cool to room temperature, maintained for 2 hours and filtered. The solid product was washed with methanol (100 ml×2) and dried at 50° C. under vacuum to obtain 2,5-dichloro-3-[5-(3,4-dimethoxy-5-nitrophenyl)-1,2,4-oxadiazol-3-yl]-4,6-dimethyl pyridine of formula (VIa) (165 g, 88%).

### Example 6: Preparation of 2,5-dichloro-3-[5-(3,4-dimethoxy-5-nitrophenyl)-1,2,4-oxadiazol-3-yl]-4,6-dimethylpyridine-1-oxide (VIIa)

To a cooled solution of 2,5-dichloro-3-[5-(3,4-dimethoxy-5-nitrophenyl)-1,2,4-oxadiazol-3-yl]-4,6-dimethylpyridine of formula (VIa) (25 g, 0.0588 mole) in 300 ml of dichloromethane was added urea hydrogen peroxide complex (18.26 g, 0.194 mole) in portions followed by trifluoroacetic anhydride (37 g, 0.176 mole) maintaining temperature below 10° C. After stirring at 5-10° C. for 1 hour, the reaction mixture was allowed to warm to room temperature and stirred for 5 hours. The reaction mixture was washed with water (300 ml×2), 300 ml of 5% aqueous sodium sulphite solution to quench residual peroxides and finally with 300 ml of water. Dichloromethane layer was distilled out at atmospheric pressure. The obtained solid was suspended in 250 ml of ethyl acetate and 12.5 ml of conc. HCl was added at room temperature. The resulting suspension was then stirred at 65-70° C. for 1 hour and allowed to cool to room temperature. After stirring for 2 hours, the reaction mixture was filtered, solid was washed with ethyl acetate (50 ml×2) followed by water (50×3) and dried at 50° C. under vacuum to obtain (5-(3,4-bis(methoxy)-5-nitrophenyl)-1,2,4-oxadiazol-3-yl)-2,5-dichloro-4,6-dimethylpyridine 1-oxide of formula (VIIa) (18 g, 69%).

### Example 7: Preparation of 5-[3-(2,5-Dichloro-4,6-dimethyl-1-oxido-3-pyridinyl)-1,2,4-oxadiazol-5-yl]-3-nitro-1,2-benzenediol (Opicapone, I)

To a cooled solution of 2,5-dichloro-3-[5-(3,4-dimethoxy-5-nitrophenyl)-1,2,4-oxadiazol-3-yl]-4,6-dimethylpyridine-1-oxide of formula (VIIa) (25 g, 0.056 mole) in 200 ml of N,N-Dimethylformamide was added AlCl₃ (11.34 g, 0.085 mol) at 5-10° C. in portions. The reaction mixture was then heated at 85° C. for 6 hours. After completion, the reaction mixture was cooled to room temperature and poured onto cold mixture of conc. HCl (200 ml) and water (400 ml). The reaction mixture was filtered, solid washed with water (100 ml×3) followed by methanol (50 ml×2) and dried at 50° C. under vacuum to obtain 5-[3-(2,5-Dichloro-4,6-dimethyl-1-oxido-3-pyridinyl)-1,2,4-oxadiazol-5-yl]-3-nitro-1,2-benzenediol of formula (I) (22 g, 94%).

### Example 8: Preparation of 2,5-dichloro-N′-hydroxy-4,6-dimethyl Nicotinimidamide of Formula (IV)

To a suspension of 2,5-dichloro-4,6-dimethylnicotinonitrile of formula (VIII) (100 g, 0.497 mole) in 1,4-dioxane (400 ml) and water (900 ml) was added 50% aqueous solution of hydroxyl amine (130 g) and N-methyl morpholine (50.2 g, 0.497) at room temperature. The reaction mixture was then stirred at 70-80° C. for 10 hours. After completion, water (1100 ml) was added to the reaction mixture at 70-80° C. and allowed to cool to room temperature. After stirring for 2 hours the reaction mixture was filtered, solid was washed with water (200 ml×3) and dried at 50° C. under vacuum to obtain 2,5-dichloro-N′-hydroxy-4,6-dimethylnicotinimidamide of formula (IV) (68 g, 58%).

### Example 9: Preparation of 2,5-dichloro-N′-hydroxy-4,6-Dimethylnicotinimidamide of Formula (IV)

To a suspension of 2,5-dichloro-4,6-dimethylnicotinonitrile of formula (VIII) (100 g, 0.497 mole) in methanol (600 ml) and water (800 ml) was added 50% aqueous solution of hydroxyl amine (130 g) and 2-methylpyrazine (7.02 g, 0.0746) at room temperature. The reaction mixture was then stirred at 70-80° C. for 6-8 hours. After completion, water (800 ml) was added to the reaction mixture at 70-80° C. and allowed to cool to room temperature. After stirring for 2 hours the reaction mixture was filtered, solid was washed with water (200 ml×3) and dried at 50° C. under vacuum to obtain 2,5-dichloro-N′-hydroxy-4,6-dimethylnicotinimidamide of formula (IV) (82 g, 70%).

### Example 10: Preparation of 2,5-dichloro-N′-hydroxy-4,6-dimethylnicotinimidamide of Formula (IV)

To a solution of hydroxylamine hydrochloride (86.4 g, 1.243 mole) in 400 ml of water was added LiOH.H₂O (52.7 g, 1.25 mole) at room temperature and heated at 50° C. for 30 minutes. To the reaction mixture was added 300 ml of methanol, 2-methylpyrazine (3.51 g, 0.037 mole) and 2,5-dichloro-4,6-dimethylnicotinonitrile of formula (VIII) (50 g, 0.248 mole) at 50° C. The reaction mixture was then stirred at 70-80° C. for 6 hours. After completion, water (500 ml) was added to the reaction mixture at 70-80° C. and allowed to cool to room temperature. After stirring for 2 hours the reaction mixture was filtered, solid was washed with water (100 ml×3) and dried at 50° C. under vacuum to obtain 2,5-dichloro-N′-hydroxy-4,6-dimethylnicotinimidamide of formula (IV) (37.6 g, 64%).

### Example 11: Purification of 5-[3-(2,5-Dichloro-4,6-dimethyl-1-oxido-3-pyridinyl)-1,2,4-oxadiazol-5-yl]-3-nitro-1,2-benzenediol (Opicapone, I)

The crude 5-[3-(2,5-Dichloro-4,6-dimethyl-1-oxido-3-pyridinyl)-1,2,4-oxadiazol-5-yl]-3-nitro-1,2-benzenediol of formula (I)(25.0 g) was suspended in 250 ml of N,N-dimethylformamide and reaction mixture was heated at 60-65° C. to obtain clear solution. Then was added 500 ml of methanol and reaction mixture was cooled to room temperature. After stirring for 2-3 hours, the reaction mixture was filtered, solid was washed with methanol and dried at 50° C. under vacuum to obtain 5-[3-(2,5-Dichloro-4,6-dimethyl-1-oxido-3-pyridinyl)-1,2,4-oxadiazol-5-yl]-3-nitro-1,2-benzenediol of formula (I) (22.0 g, 88%).

We claim:

1. A process for the preparation of opicapone compound of formula (I) comprising steps of;
a) reacting compound of formula (II) with oxone to obtain compound of formula (III); 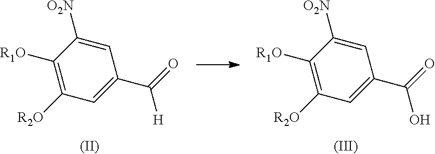
b) reacting compound of formula (III) with compound of formula (IV) to obtain compound of formula (V); 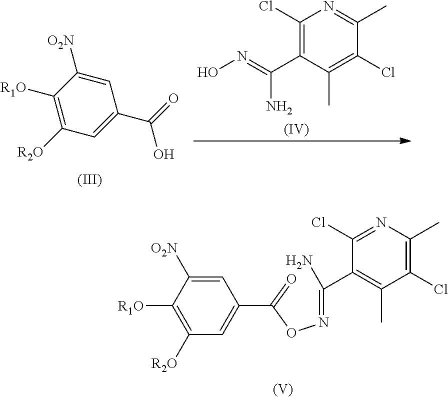
c) optionally isolating compound of formula (V);
d) cyclizing compound of formula (V) in presence of base at room temperature to obtain compound of formula (VI); 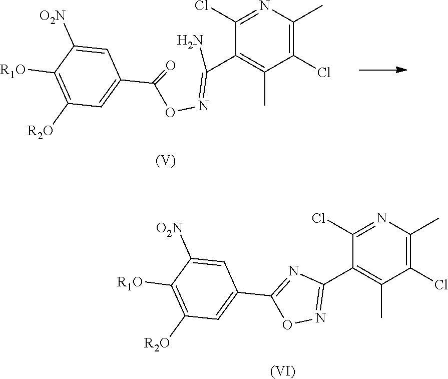
e) oxidizing compound of formula (VI) with oxidizing agent to produce compound of formula (VII); 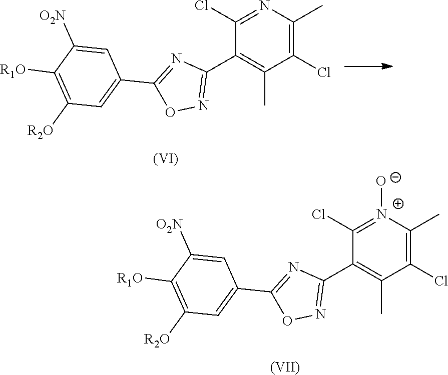
f) deprotecting hydroxyl protecting group of compound of formula (VII) to obtain opicapone compound of formula (I); 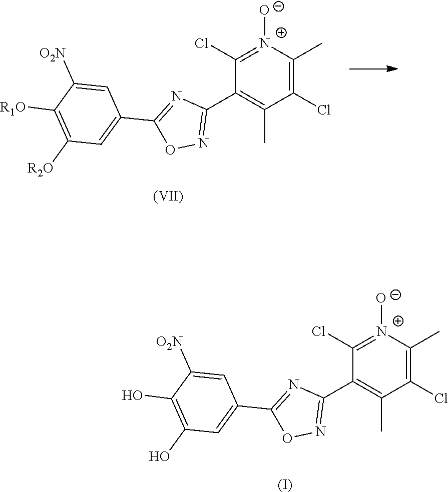
wherein R₁ and R₂ independently from each other represent hydrogen or a suitable protecting groups for aromatic hydroxyl groups.

2. The process according to claim 1, wherein suitable protective groups for aromatic hydroxyl groups are selected from methyl, ethyl, isopropyl, butyl, benzyl, 4-methoxybenzyl, methoxymethyl, benzyloxymethyl, methoxyethoxymethyl, tetrahydropyranyl, phenacyl, allyl, trimethylsilyl, tert-butyldimethylsilyl, benzyloxycarbonyl, tert-butoxycarbonyl, ester, sulphonate, carbamate, phosphinate, acetal, ketal derivatives.

3. The process according to claim 1 wherein step (a) is performed in solvent selected from N, N-Dimethylmethanamide (DMF), acetone, acetonitrile, N-methylpyrrolidone (NMP), Hexamethylphosphoramide (HMPA), pyrrolidinone, tetrahydrofuran, water, thyl acetate, 1,4-dioxane, acetonitrile, propionitrile, acetone, ethyl methyl ketone, formamide, dichloromethane, ethylene dichloride, chloroform, dimethyl acetamide, propionamide, nitromethane, 1,2-dimethoxyethane, 2-methoxyethanol, 2-ethoxy ethanol, aliphatic, hexane, heptane, pentane, cyclohexane, methyl cyclohexane, aliphatic esters and mixture(s) thereof.

4. The process according to claim 1 wherein step (b) is performed in presence of condensing agent selected from group consisting of N, N′-Carbonyldiimidazole, thionyl chloride, sulfonylchloride, N,N′-dicyclohexylcarbodiimide, 1-hydroxybenzotriazole and N-(3-dimethylaminopropyl)-N′-ethylcarbodiimide, phosgene, PCl3, POCl3, PCl5, anhydrides, trichlorotriazine and chlorodimethoxytriazine.

5. The process according to claim 1 (d) wherein base is selected from group consisting of triethyl amine, diisopropylethylamine, DMAP, sodium hydroxide (NaOH), potassium hydroxide (KOH), potassium carbonate, sodium carbonate, sodiumbicarbonate, potassium bicarbonate, sodium hydride, potassium ter-butoxide, sodium ter-butoxide and/or and aqueous(s) solution and mixture(s) thereof.

6. The process according to claim 1 (e) wherein oxidizing agent is selected from the group consisting of peroxide, MnO₂, peracetic acid, trifluoroperacetic acid, t-butylhydroperoxide, m-chloroperoxybenzoic acid, persulfuric acids, Oxone®, urea hydrogen peroxide complex and trifluoroacetic anhydride, pyridinium chlorochromate and permanganate ions.

7. The process according to claim 1 (f) wherein deprotection is carried out in presence of aluminium chloride (AlCl3) in organic solvent.

8. A compound of formula (Va), (VIa) and VIIa) useful for the preparation of opicapone compound of formula (I).

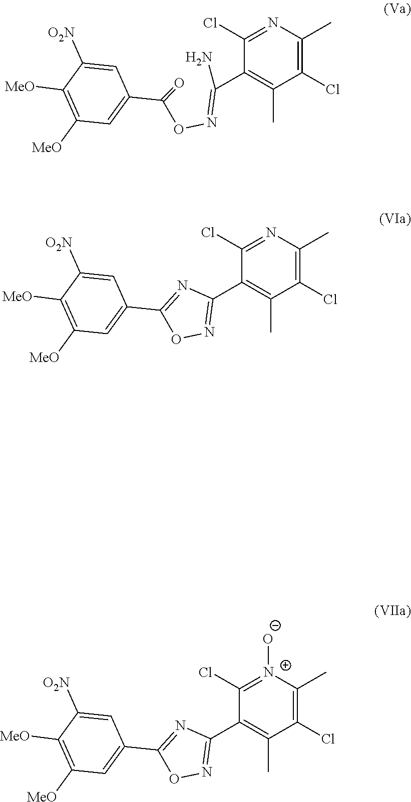 [SMILES: COc1cc(C(=O)O/N=C(\N)c2c(Cl)nc(C)c(Cl)c2C)cc([N+](=O)[O-])c1OC ; COc1cc(-c2nc(-c3c(Cl)nc(C)c(Cl)c3C)no2)cc([N+](=O)[O-])c1OC ; COc1cc(-c2nc(-c3c(C)c(Cl)c(C)[n+]([O-])c3Cl)no2)cc([N+](=O)[O-])c1OC]

9. The process according to claim 1, wherein opicapone further comprising a purification step of opicapone compound of formula (I) with a organic solvent selected from methanol, ethanol, isopropyl alcohol, dichloromethane, tetrahydrofuran, toluene, N,N-dimethylformamide, dimethylsulfoxide, N,N-dimethylacetamide, N-methyl-2-pyrrolidone, acetic acid, ethylacetate, acetone, and mixture(s) thereof.

10. A process for the preparation of compound of formula (IV) comprising;
a. reacting compound of formula (VIII) with hydroxyl amine in the presence of catalytic amount of pyrazine or pyrazine derivative;
OR
b. reaction compound of formula (VIII) with hydroxyl amine or its salt thereof in the presence of base and catalytic amount of pyrazine or pyrazine derivative.
